(19) United States
(12) Patent Application Publication
(10) Pub. No.: US 2023/0355219 A1
(43) Pub. Date: Nov. 9, 2023

(54) SPECIMEN TRANSPORT MEDIUM TUBE

(71) Applicant: Mingfu LING, Oakville, Ontario (CA)

(72) Inventor: Mingfu LING, Oakville, Ontario (CA)

(21) Appl. No.: 18/247,194

(22) PCT Filed: Oct. 8, 2021
(86) PCT No.: PCT/CA2021/051421

§ 371 Date: Mar. 29, 2023

Related U.S. Application Data
(60) Provisional application No. 63/090,167, filed on Oct. 9, 2020.
(60) Provisional application No. 63/160,702, filed on Mar. 12, 2021.

(51) Int. Cl. A61B 10/00; B01L 3/00

(52) U.S. Cl. CPC A61B 10/0096; A61B 10/0045; A61B 10/0051; B01L 3/5029; B01L 2200/185; B01L 2300/0832; B01L 2300/042

(57) ABSTRACT

A specimen collection kit includes a specimen transport medium tube, a transport medium and a swab for collecting a sample. The transport medium compartment of the specimen transport medium tube has a reduced inner diameter and contains a reduced volume of the transport medium compared to conventional transport medium tubes. The tip of a swab containing a collected sample which is stored in the specimen transport medium tube can remain immersed in the transport medium during transport to a testing facility, preventing drying of the swab tip and degradation of the sample. In addition, the relatively small volume of transport medium in the tube reduces the dilution of analytes from the sample in the a transport medium.

### BACKGROUND

The present application is directed to devices for storing and transporting collected samples. More specifically, the present application is directed to improved specimen transport medium tubes for storing and transporting samples collected on swabs, and to specimen collection kits containing such tubes.

Specimens or samples are routinely required for predicting or diagnosing a disease or condition in a subject in need of care thereof. The samples may be collected in a medical facility (for example, a hospital or clinic) or in any number of environments, for example, in the subject's home, workplace or community. Specimens, such as from buccal surfaces, nasopharynx, wounds, blood spatter or drops, etc., can be collected with absorbent material (for example, nylon) from the tip of a swab. Nasopharyngeal swabs have been used to collect samples from the nasopharynx to test for the presence of COVID-19 and influenza viruses, etc. Oropharyngeal swabs have also been used to collect samples from the oropharynx to test for COVID-19 and influenza viruses, etc. The collected swab (particularly the swab tip) is then dipped into a transport medium in a closed receptacle in the form of a tube to preserve the collected specimen. The collected swab immersed in the transport medium in a transport medium tube is then transported to a laboratory where the lab tests are to be performed. The specimen collected on the swab tip in the tube is eluted into the transport medium. Once received in the laboratory, the transport medium is subjected to various laboratory testing. Depending upon the specific purpose of the lab testing, the presence or the concentration of analytes, RNA, DNA, proteins, lipids, carbohydrates and other molecules, or the quantification or identification of live viruses, bacteria, fungi and other microbes may be determined.

Currently, in order to collect a sufficient amount of specimen from a subject, the swab tip needs not only to be made from an absorbent material, but also to have sufficient surface area to collect the specimen (for example, the length of a swab tip is generally between 10 and 25 mm and the diameter of a swab tip is generally between 3 and 6 mm). Because the swab tip needs to be fully immersed in a transport medium in the tube to preserve the specimen, 1 ml, 2 ml, or 3 ml of the transport medium is currently needed in the sample collection system. The required volume of the transport medium is dictated by the diameter of the transport medium tube. Table 1 provides examples of transport medium volumes of commercial universal transport medium (UTM) products.

TABLE 1
A survey of transport medium volume among commercial
universal transport medium products

| Transport medium | Volume | |
|---|---|---|
| Copan UTM ™ | 3 | mL |
| Becton Dickinson UVT | 1 ml or 3 mL | |
| Hardy Diagnostics HealthLink ™ UTM ™ | 3 | mL |
| Longhorn Vaccines and Diagnostics PrimeStore ® MTM | 1.5 | mL |

In addition, in order to encompass the length of swab inside the transport medium tube, known transport medium tubes need to be long. For example, Copan's UTM® 302C Viral Transport Medium offers 1 ml or 3 ml transport medium in a 16 mm diameter×100 mm length tube, providing a volume of interior chamber space of about 20 ml that includes air space plus the transport medium volume. In such a transport medium tube, a large in-tube air space (17 ml for 3 ml transport medium or 19 ml for 1 ml transport medium) exists above the transport medium. However, during transport of the transport medium tube, in particular by mail or courier services, it is very likely that the tube will be placed on its side or even upside down, with the effect that the collected specimen swab tip may not be immersed in the transport medium for hours or even longer. If the swab tip is not immersed in the transport medium during transportation or storage, dehydration of the collected specimen swab tip and degradation of pathogen specimen may occur, leading to false negative results.

In contrast, current molecular diagnostic tests or assays including nucleic acid detection only need or use a small fraction of the collected transport medium volume. For example, most molecular diagnostic tests (DNA detection assays and RNA detection assays by PCR or RT-PCR) only use up to 200 μI or even less (20-100 μl) of the collected transport medium (see Table 2). Moreover, nucleic acids isolated from this 20-200 μl of collected transport medium can even carry 3 or more repeats for nucleic acid detection assays.

TABLE 2
A survey of sample input volumes
among DNA/RNA isolation methods

| | Nucleic | | |
|---|---|---|---|
| Method | Acid Type | Sample Volume | |
| QIAamp ™ DNA Mini Kit | DNA | 200 | μl |
| QIAamp ® Viral RNA Mini Kit | RNA | 140 | μl |
| ThermoFisher KingFisher ™ | DNA or RNA | 20 to 200 | μL |
| Nucleic Acid | | | |
| Purification Systems | | | |

Not only molecular diagnostic tests (PCR based methods), but also virological or microbiological studies also require small volumes of collected transport medium for lab testing. For example, only up to 200 μl is needed for plating or inoculating for detection of Chlamydia trachomatis and Neisseria gonorrhoeae. In summary, there is typically no need for more than 200 μl of collected transport medium for lab testing. Thus, a major issue with collecting the swab in a volume of 1-3 ml transport medium, rather than the required testing volume of 100-200 μl, is that the specimen collected on the swab is unnecessarily diluted in the transport medium, resulting in a low titer of pathogen specimen, which may result in concentrations or amounts below the detection limit of the current assay, thus leading to further false negative results.

It is evident that this problem cannot be solved by simply reducing the volume of the transport medium placed in conventional prior-art transport medium tubes, featuring diameters greater than 10 mm, and typically 16 mm. As shown in Table 3 below, a minimum of 2 ml of transport medium is needed in a conventional prior-art transport medium tube to reach 11.3 mm medium fill height in order to cover a 10 mm length of a swab tip.

TABLE 3
Medium Fill Heights of Various Medium Volumes
in a Prior Art 15 mm Inner Diameter Tube

| Volume (mL) | 0.4 | 0.6 | 0.8 | 1 | 2 |
|---|---|---|---|---|---|
| Medium Fill | 2.3 | 3.4 | 4.5 | 5.7 | 11.3 |
| Heights (mm) | | | | | |

Because the swab tip is typically between 10-25 mm in length, if a prior art sample collection tube were used with a smaller volume of a transport medium (e.g., less than 2 ml), the height of the transport medium would not be sufficient to cover most or the entire length of a swab tip even when the tube is held upright. If the tube should be laid on its side, there would be even less medium coverage for the swab tip. Thus, the swab, and the sample collected on the swab, would be at risk of dehydration and degradation.

Accordingly, a need exists to address the above-discussed problems and improve both the design of specimen collection kits and the method or process of specimen collection to facilitate the diagnosis of a disease or condition in a subject in need thereof.

### SUMMARY

In one aspect, the present invention provides a specimen transport medium tube for transporting a sample disposed on a swab tip of a swab. The specimen transport medium tube includes a mouth end with an opening configured to receive the swab tip, a distal end opposite the mouth end to define a longitudinal axis of the specimen transport medium tube, and at least one side wall extending longitudinally between the mouth end and the distal end to define and enclose an interior space inside the specimen transport medium tube. The interior space is in fluid communication with the opening of the mouth end. The tube also includes a cap configured to engage the mouth end so as to seal the opening.

The interior space of the specimen transport medium tube includes a mouth portion at the mouth end, a transport medium compartment longitudinally aligned with the longitudinal axis of the tube, and a tapered portion disposed between the mouth portion and the transport medium compartment, which is in fluid communication with both the mouth portion and with the transport medium compartment. A swab tip may be passed without hindrance through the opening into the mouth portion and through the tapered portion to be contained in the transport medium compartment. The transport medium compartment can also contain a transport medium. The cap includes a cap insert which is configured to prevent fluid communication between the mouth portion and the transport medium compartment when the cap engages the mouth end.

The inner diameter of the transport medium compartment is smaller than the inner diameter of the mouth portion. The inner diameter of the tapered portion decreases from the inner diameter of the mouth portion at a junction between the tapered portion and the mouth portion to the inner diameter of the transport medium compartment at a junction between the tapered portion and the transport medium compartment. In at least one embodiment, the inner diameter of the transport medium compartment is less than or equal to 10 mm. In at least one embodiment, the inner volume of the transport medium compartment is less than 1 ml.

Another aspect of the present invention provides a specimen collection kit comprising a specimen transport medium tube as described herein, a transport medium and a swab. In at least one embodiment, the kit further contains instructions for use of the kit. In at least one embodiment, the transport medium is contained within the transport medium compartment of the specimen transport medium tube. In at least one embodiment, the swab is affixed to the cap of the specimen transport medium tube. In at least one embodiment, the swab is a nasopharyngeal swab. In at least one embodiment, the swab is an oropharyngeal swab.

### BRIEF DESCRIPTION OF THE DRAWINGS

Further features of the present invention will become apparent from the following written description and the accompanying figures, in which:

FIG. 1A is a side view of an embodiment of a specimen collection kit according to the present application.

FIG. 1B is a side view of an alternative embodiment of a specimen collection kit according to the present application.

FIG. 1C is a side view of an alternative embodiment of a specimen collection kit according to the present application.

FIG. 2A is a side view of an alternative embodiment of a specimen collection kit according to the present application.

FIG. 2B is a side view of an alternative embodiment of a specimen collection kit according to the present application.

FIG. 2C is a side view of an alternative embodiment of a specimen collection kit according to the present application.

FIG. 2D is a side view of an alternative embodiment of a specimen collection kit according to the present application.

FIG. 3A is a side view of the embodiment of FIG. 1A in use.

FIG. 3B is an alternative side view of the embodiment of FIG. 1A in use.

FIG. 3C is an alternative side view of the embodiment of FIG. 1A in use.

FIG. 4A is an alternative side view of the embodiment of FIG. 1A in use.

FIG. 4B is an alternative side view of the embodiment of FIG. 1A in use.

FIG. 4C is an alternative side view of the embodiment of FIG. 1A in use.

FIG. 5A is a side view of the embodiment of FIG. 2A in use.

FIG. 5B is an alternative side view of the embodiment of FIG. 2A in use.

FIG. 5C is an alternative side view of the embodiment of FIG. 2A in use.

FIG. 5D is an alternative side view of the embodiment of FIG. 2A in use.

FIG. 6A is a side view of an alternative embodiment of a specimen collection kit according to the present application.

FIG. 6B is a side view of the embodiment of FIG. 6A in use.

FIG. 6C is an alternative side view of the embodiment of FIG. 6A in use.

FIG. 6D is an alternative side view of the embodiment of FIG. 6A in use.

FIG. 6E is an alternative side view of the embodiment of FIG. 6A in use.

FIG. 6F is an alternative side view of the embodiment of FIG. 6A in use.

FIG. 7A includes a side view, a perspective view, a top view, a bottom view and a cross-sectional view of the cap insert head of the embodiment of FIG. 6A.

FIG. 7B is a top view of the cap insert head of the embodiment of FIG. 6A enclosing a swab handle in an uncompressed state.

FIG. 7C is a top view of the cap insert head of the embodiment of FIG. 6A enclosing a swab handle in a compressed state.

FIG. 8A is a side view of an alternative embodiment of a specimen collection kit according to the present application.

FIG. 8B is a side view of the embodiment of FIG. 8A in use.

FIG. 8C is an alternative side view of the embodiment of FIG. 8A in use.

FIG. 8D is an alternative side view of the embodiment of FIG. 8A in use.

FIG. 8E is an alternative side view of the embodiment of FIG. 8A in use.

FIG. 9A includes a side view, a perspective view, a top view, a bottom view and a cross-sectional view of the cap insert head of the embodiment of FIG. 8A.

FIG. 9B is a top view of the cap insert head of the embodiment of FIG. 8A enclosing a swab handle in an uncompressed state.

FIG. 9C is a top view of the cap insert head of the embodiment of FIG. 8A enclosing a swab handle in a compressed state.

### DETAILED DESCRIPTION

In at least one embodiment, the present invention provides a specimen collection kit for collecting, storing, preserving and/or transporting a collected specimen or sample. The specimen can be any sample which may be collected on a swab. For example, samples may be collected by contacting a surface with a swab so as to transfer material from the surface to the swab tip, or exposing a swab to a fluid so that a portion of the fluid is absorbed on the swab tip. Surfaces which may be sampled include but are not limited to surfaces in environmental or industrial settings, such as the home, workplace or community, or surfaces found on or in biological organisms, including but not limited to mucosal surfaces such as oral, buccal, nasal, pharyngeal, aural, urethral, anal and vaginal surfaces, skin, and other surfaces. Fluids which may be sampled include but are not limited to fluids found in environmental or industrial settings, such as the home, workplace or community, fluids exposed to or containing biological material, including but not limited to microorganisms, and fluids originating from biological organisms or microorganisms, including but not limited to secretions from mucosal surfaces or orifices or from a wound, blood, urine and other biological fluids or secretions.

In at least one embodiment, samples may be obtained for laboratory testing, including but not limited to analytical, microbiological, molecular diagnostic and other examinations. Such testing includes but is not limited to testing for the presence or the concentration or amount of analytes. As used herein, the term “analyte” is intended to mean any material or substance whose presence, amount or concentration is subject to measurement and includes but is not limited to cells; microorganisms, including but not limited to viruses, bacteria, fungi and other microorganisms; and molecular analytes including but not limited to RNA, DNA, proteins, lipids, carbohydrates and other chemical substances.

The specimen collection kit includes a specimen transport medium tube, a transport medium and a swab for collecting specimens or samples. The swab has an elongate swab handle which is attached at at least one end to a swab tip, and can be a conventional swab as well known in the art. In at least embodiment, the swab handle is cylindrical or prismatic. In at least one embodiment, the length of the swab, including the handle and swab tip, ranges from about 7.5 cm to about 15 cm, as needed to allow a user to conveniently place the swab tip in position for collecting a desired sample within a site of limited accessibility, including but not limited to the interior lumen of a nose, mouth, pharynx, ear, rectum, urethra, vagina or other orifice. In at least one embodiment, the swab handle is made of plastic, wood, paper, glass or metal. In at least one embodiment, the swab handle is made of a resilient material, including but not limited to plastic materials such as polypropylene, polystyrene, or acrylonitrile butadiene styrene (ABS) plastic.

In at least one embodiment, the swab handle has a narrowed region or breakpoint which facilitates breaking the handle so that a shortened swab, including the swab tip and a shortened portion of the handle attached to the swab tip, can be separated from the remainder of the handle distal to the swab tip. In at least one embodiment, the breakpoint is located about 1 cm to about 9 cm from the end of the swab at the swab tip, so that after breakage of the handle at the breakpoint, the net length of the shortened swab can range from about 1 cm to about 9 cm. In at least one embodiment, the breakpoint is located about 3 cm to about 9 cm from the end of the swab at the swab tip, so that after breakage of the handle at the breakpoint, the net length of the shortened swab can range from about 3 cm to about 9 cm. As used herein, the term “net length of the swab” is intended to mean the total length of the swab tip and the attached handle or the total length of the swab tip and the attached portion of the broken handle after breakage of the handle at the breakpoint. In at least one embodiment, the breakpoint can be located about 3 cm from the end of the swab at the swab tip, such that the net length of the swab is about 3 cm. In at least one embodiment, the breakpoint can be located about 7.5 cm to about 9 cm from the end of the swab at the swab tip, such that the net length of the swab is about 7.5 cm to about 9 cm.

In at least one embodiment, the swab tip is made from a material suitable for absorbing or incorporating desired samples, as would be understood in the art. In at least one embodiment, the materials are porous or fibrous. In at least one embodiment, the materials are hydrophilic so as to readily absorb or incorporate aqueous or other hydrophilic samples during collection. In at least one embodiment, the materials are hydrophobic, so any collected hydrophilic samples are more readily released from the swab tip and solubilized into a hydrophilic transport medium in which the swab tip is immersed. The person of skill in the art would be readily able to select a swab having an appropriate material at the swab tip for a particular application. Suitable materials for the swab tip include but are not limited to cotton, cotton-like material, rayon, wool, nylon, porous plastic sponge material including but not limited to polyethylene, and other materials well known in the art. The swab tip is attached to the handle by any means known in the art. For example, the material making up the swab tip can be wound around the handle or attached to the handle by flocking or with an adhesive.

In at least one embodiment, the specimen transport medium tube includes a mouth end, a distal end opposite the mouth end and defining a longitudinal axis therewith, and at least one side wall extending longitudinally between the mouth end and the distal end to form a tubular structure, thereby defining and enclosing an interior space inside the specimen transport medium tube. Each of the at least one side wall of the specimen transport medium tube may be planar or arcuate, such that the specimen transport medium tube has a round or polygonal cross-section at any point along its length, as long as the interior space of the specimen transport medium tube is enclosed by the mouth end, the distal end and the at least one side wall. In at least one embodiment, the mouth end includes an opening which is configured to receive a swab containing a collected sample. In at least one embodiment, the interior space includes a mouth portion at the mouth end of the tube which is in fluid communication with the opening at the mouth end, so as to allow the free flow of a gas, liquid or other fluid therebetween.

In at least one embodiment, the interior space further includes a transport medium compartment configured to contain the transport medium, as will be more fully described below, and having an inner diameter which is smaller than the inner diameter of the mouth portion. The transport medium compartment of the interior space is longitudinally aligned within the longitudinal axis of the specimen transport medium tube, and has a first end in fluid communication with the mouth end of the tube, and a second end defined by the distal end of the tube. At least one gradually sloping or stepped side wall extends between at least one side wall of the mouth portion and at least one side wall of the transport medium compartment, to form a tapered or stepped portion of the interior space of the tube having a transverse diameter or width which gradually decreases between the mouth portion and the first end of the transport medium compartment. In at least one embodiment, the inner diameter of the mouth end of the specimen transport medium tube is from about 18 mm to about 21 mm. In at least one embodiment, the inner diameter of the transport medium compartment is less than about 10 mm. In at least one embodiment, the mouth portion and transport medium compartments of the specimen transport medium tube may be cylindrical or prismatic in shape, while the tapered or stepped portion may be conical or polyhedral in shape.

In at least one embodiment, the distal end of the specimen transport medium tube includes a distal end wall which is perpendicular to the longitudinal axis of the specimen transport medium tube and unitary with the at least one side wall at the distal end of the specimen transport medium tube, such that the distal end of the tube is closed and prevents fluid communication between the exterior of the tube and the interior space of the tube, including the transport medium compartment. In at least one embodiment, the distal end of the specimen transport medium tube has an opening permitting fluid communication between the exterior of the tube and interior space of the tube, including the transport medium compartment. In such embodiments, the specimen transport medium tube may further include a removable and replaceable distal end cap which engages the one or more side walls at the distal end of the tube, thereby acting to seal the opening at the distal end of the tube, preventing loss of transport medium from the transport medium compartment during transport of the tube, for example. Removal of the distal end cap can facilitate retrieval of the transport medium from the transport medium compartment for analysis, for example. In certain embodiments, the distal end cap may engage the one or more side walls at the distal end of the tube by way of a helical screw thread configured to mate with a complementary helical screw thread located on the one or more side walls at the opening at the distal end of the specimen transport medium tube. In at least one alternative embodiment, the distal end cap may snap snugly over the one or more side walls at the opening, insert snugly within the opening, or engage the one or more side walls at the distal end of the tube and seal the opening by other methods well known in the art. Suitable materials for the distal end cap include but are not limited to rubber, silicone and moldable plastic materials, as well known in the art, including but not limited to polystyrene, polypropylene and other moldable plastic materials.

It will be evident to the skilled person, in view of the reduced inner diameter of the transport medium compartment compared to the inner diameter of the mouth portion of the specimen transport medium tube, that in at least one embodiment, the distal end of the specimen transport medium tube may have a width or diameter smaller than the width or diameter of the mouth end of the tube. In at least one such embodiment, the specimen transport medium tube may include a support structure at its distal end, to provide greater stability to the specimen transport medium tube when resting upright on its distal end. In at least one embodiment, the support structure includes a flat base having a width or diameter larger than the width or diameter of the distal end of the specimen transport medium tube and oriented perpendicular to the longitudinal axis of the specimen transport medium tube. In at least one embodiment, the support structure may be unitary with the at least one side wall at the distal end of the specimen transport medium tube. In at least one embodiment, the support structure may be unitary with, or fixed to the distal end cap. Suitable materials for the support structure include but are not limited to glass and moldable plastic materials, as well known in the art, including but not limited to polypropylene and other moldable plastic materials.

In at least one alternative embodiment, the specimen transport medium tube may include one or more additional external side walls extending parallel to the longitudinal axis from the mouth end of the tube to the distal end of the tube. In at least one embodiment, the at least one external side wall can provide a surface on which sample identification details can be recorded by writing or affixing a label, or on which a barcode label may be affixed, for example. In at least one embodiment, the at least one external side wall may facilitate handling of the specimen transport medium tube by automated robotic liquid handling systems. For example, in at least one embodiment, the at least one external side wall can provide a surface which facilitates gripping of the specimen transport medium tube by a robotic arm.

In at least one embodiment, the one or more external side walls end at or beyond the distal end of the tube to form an opening, such that the distal end of the tube and the distal end cap, if present, are accessible external to the tube. In at least one embodiment, the opening formed by the external side walls at the distal end of the tube has a width or diameter substantially equal to the cross-sectional width or diameter of the tube at the mouth end and is parallel therewith, and thus, the ends of the one or more external side walls define a planar surface which is perpendicular to the longitudinal axis of the specimen transport medium tube and can act as a support structure for the specimen transport medium tube, permitting the tube to be supported upright at its distal end. In at least one embodiment, the ends of the one or more external side walls can be enclosed by an external distal end wall extending perpendicular to the longitudinal axis of the specimen transport medium tube to form a flat support surface enclosing the distal end of the specimen transport medium tube, again permitting the tube to be supported upright at its distal end. In at least one embodiment, the flat support surface formed by the external distal end wall at the distal end of the tube has a width or diameter substantially equal to the width or diameter of the tube at the mouth end, and can act as a support structure for the specimen transport medium tube.

In at least one embodiment, the specimen transport medium tube includes a removable and replaceable cap configured to engage the one or more side walls at the mouth end of the tube, thereby to seal the opening at the mouth end of the tube. In at least one embodiment, the cap includes a helical screw thread configured to mate with a complementary helical screw thread located on the one or more side walls at the opening at the mouth end of the specimen transport medium tube, thereby to seal the opening as is well known in the art. In at least one alternative embodiment, the cap may snap snugly over the one or more side walls at the opening, insert snugly within the opening, or otherwise seal the opening, as will be well understood by the skilled person. Suitable materials for the cap include but are not limited to moldable plastic materials, as well known in the art, including but not limited to polypropylene and polyethylene terephthalate.

In at least one embodiment, the cap includes a cap insert which is inserted into the mouth portion of the tube when the cap engages the one or more side walls at the mouth end of the specimen transport medium tube. The cap insert may be fixed to or unitary with the cap or may engage with the cap when the cap is secured over the opening at the mouth end of the specimen transport medium tube. If the cap insert is fixed to or unitary with the cap, it should be configured so as not to impede the engagement of the cap with the one or more side walls at the at the mouth end of the tube. In at least one embodiment, the cap insert is inert towards the transport medium, such that it does not significantly degrade in contact with the transport medium and does not cause significant degradation of the transport medium. Suitable materials for the cap insert include but are not limited to silicone, rubber or plastic, including but not limited to polypropylene and polyethylene. The cap insert may be manufactured using plastic injection molding techniques, as well known in the art.

The cap insert can include a cap insert head which, when the cap is secured over the opening to the mouth portion of the specimen transport medium tube, seats snugly within the tapered or stepped portion of the tube, or against or within the opening of the transport medium compartment into the tapered or stepped portion of the specimen transport medium tube, so as to seal the interface between the transport medium compartment and the tapered or stepped portion of the specimen transport medium tube, and prevent the transport medium contained in the transport medium compartment from escaping or leaking from the transport medium compartment of the tube. In at least one embodiment, the cap insert head has a contour which complements the contour of the interior of the tapered or stepped portion of the specimen transport tube, so as to snugly seat thereto.

In at least one embodiment, the cap insert head is made of a material which is inert towards the transport medium, such that it does not significantly degrade in contact with the transport medium and does not cause significant degradation of the transport medium, and which is resiliently compressible so as to conform to the shape of the tapered or stepped portion of the tube or fit snugly within the opening of the transport medium compartment, and to fill any gaps or air spaces which might otherwise permit flow of transport medium, thereby sealing the interface between the transport medium compartment and the tapered or stepped portion of the specimen transport medium tube. As used herein, the term “resiliently compressible” is intended to mean that the material is flexible and can be reduced in volume when subjected to pressure, but is resilient in its compressed state so as to fully occupy the reduced volume available to it.

The cap insert head can assume a variety of shapes as long as the cap insert head acts to seal the interface between the transport medium compartment and the tapered or stepped portion of the specimen transport medium tube, and prevent the transport medium contained in the transport medium compartment from escaping the transport medium compartment and entering the mouth portion of the tube. In at least one embodiment, the cap insert head includes an O-ring or gasket mounted on the cap insert, such that the O-ring or gasket fits snugly within the tapered or stepped portion of the interior space of the tube or within the opening at the first end of the transport medium compartment. In at least one embodiment, the O-ring is positioned about 1 mm to about 7 mm above the distal end of the cap insert, such that it snugly contacts the inner side wall of the tapered or stepped portion of the specimen transport medium tube.

The cap insert head may be manufactured using plastic injection molding techniques, as well known in the art. When the cap insert head includes an O-ring, the O-ring may be manufactured by extrusion, injection molding, pressure molding or transfer molding, as known in the art. Suitable materials for the cap insert head include but are not limited to silicone, rubber, vulcanized material or plastic, including but not limited to polypropylene. The cap insert and cap insert head can vary in shape and can be solid or hollow in the interior thereof, as long as the cap insert head can be positioned to seal the opening of the transport medium compartment and prevent loss of the transport medium therefrom.

In use, the larger diameter of the mouth portion and the presence of the tapered portion allow a user to more easily insert a swab tip containing a collected sample into the specimen transport medium tube and guide the swab tip into position in the transport medium compartment. Once the swab tip is placed in the transport medium compartment containing the transport medium, the handle may be conveniently broken at the breakpoint or cut at any convenient point on the handle to form a shortened swab, so that the swab tip in the transport medium compartment is attached to only the short portion of the handle retained with the swab tip after breakage. The cap can then be secured on the mouth portion of the specimen transport medium tube such that the cap seals the opening at the mouth end of the tube and the cap insert head seals the interface between the transport medium compartment and the tapered or stepped portion of the specimen transport medium tube. In at least one embodiment, the cap is secured on the mouth portion of the specimen transport medium tube by engaging a helical screw thread on the cap with a complementary helical screw thread on the one or more side walls of the mouth portion.

The specimen transport medium tube, therefore, in at least one embodiment, has a length which is sufficient to include the net length of the swab. In at least one embodiment, the length of the specimen transport medium tube will exceed the net length of the swab by at least 5 mm. In this way, the net length of the swab can be fully contained within the specimen transport medium tube. In at least one embodiment, the length of the specimen transport medium tube is from about 90 mm to about 95 mm.

It is contemplated that the portion of the swab handle retained with the swab tip in the swab or shortened swab may be fully contained within the transport medium compartment, or the portion of the swab handle may extend into the mouth portion of the tube. In certain embodiments where the swab handle extends into the mouth portion of the tube, the cap insert head may displace the handle towards the inner side wall of the tapered or stepped portion of the tube when in place and the cap insert head may deform around the handle, so as to seal the opening of the transport medium compartment. In embodiments where the cap insert head is an O-ring, the O-ring desirably has a cross-sectional diameter of at least 1.5 mm or at least 2 mm, to have an adequate resilient compressibility to deform around the handle and seal the opening of the transport medium compartment.

In certain embodiments, the cap insert head may have a groove or slit to snugly encompass the swab handle, allowing sealing of the opening of the transport medium compartment. The cap insert may further include a bracket or clip to retain the swab handle, as discussed in further detail below. Such embodiments are adapted to accommodate a swab having a resilient handle or a handle with a smaller diameter along at least part of the length of the handle adjacent to the swab tip, such as a nasopharyngeal swab.

In at least one alternative embodiment where the swab handle extends into the mouth portion of the tube, the cap insert may include a central longitudinal channel through which the swab handle can pass, and which forms a seal around the swab handle to prevent transport medium from passing from the transport medium compartment through the central longitudinal channel into the mouth portion of the specimen transport medium tube. Such embodiments are adapted to accommodate a swab, having a less resilient handle or a handle with a larger diameter, such as an oropharyngeal swab.

As used herein, the term “transport medium” is intended to mean a fluid in which a swab tip is placed after the swab has been used to collect a sample or specimen. In at least one embodiment, the transport medium provides an environment in which any microbes or cells present on the swab tip may be kept alive for further culture or growth. In at least one embodiment, the transport medium provides an environment in which any microbes or cells present on the swab tip may be preserved in a live, dead or dormant state for further examination or analysis. In at least one embodiment, the transport medium provides an environment which prevents analytes present on the swab tip from undergoing degradation or decomposition.

In at least one embodiment, the transport medium is aqueous. In at least one embodiment, the transport medium may contain additives, including but not limited to nutrients, buffers, salts, antimicrobials, preservatives, reagents, and other additives well known in the art. Suitable preservatives for preserving biodegradable analytes such as proteins, DNA and RNA include but are not limited to denaturants, including but not limited to sodium dodecyl sulfate (SDS) and chaotropic agents including but not limited to guanidinium hydrochloride, guanidinium thiocyanate and alcohols. Suitable transport media are well known in the art and/or commercially available and include but are not limited to saline, phosphate-buffered saline, universal transport medium, viral transport medium, guanidine-containing medium, propagating transport medium, non-propagating transport medium, anaerobic transport medium, charcoal transport medium, bacterial transport medium and other transport media well known in the art.

The transport medium compartment of the present specimen transport medium tube has an inner diameter which is reduced compared to the inner diameter of conventional transport medium tubes. In this way, the volume of transport medium accommodated within the transport medium compartment is relatively small, so as not to overly dilute the sample collected on the swab tip, but sufficient to immerse the entire length of the swab tip, so as to keep the collected sample exposed to the transport medium regardless of the gravitational orientation or position of the specimen transport medium tube during transport. Table 4 provides examples of volumes of transport medium needed in transport medium compartments with various inner diameters in order to achieve medium fill heights sufficient to immerse swab tips of various lengths.

TABLE 4
Volume of Transport Medium Needed to Achieve 10, 15, 20 and 25
mm of Medium Fill Heights with Various Tube Inner Diameters

| Inner diameter of the | Volume of transport medium required (mL) | | | |
|---|---|---|---|---|
| compartment (mm) | to achieve a medium fill height of | | | |
| transport medium | 10 mm | 15 mm | 20 mm | 25 mm |
| 9 | 0.64 | 0.95 | 1.27 | 1.59 |
| 8 | 0.50 | 0.75 | 1.00 | 1.26 |
| 7 | 0.38 | 0.58 | 0.77 | 0.96 |
| 6 | 0.28 | 0.42 | 0.57 | 0.71 |
| 5 | 0.20 | 0.29 | 0.39 | 0.49 |
| 4 | 0.13 | 0.19 | 0.25 | 0.31 |

Thus, for example, for a transport medium compartment with 9 mm inner diameter, only 0.64 ml of transport medium is required in order to have a medium fill height of 10 mm. Alternatively, as much as 1.59 ml of transport medium is required to provide a medium fill height of 25 mm. In an additional example, for a transport medium compartment with 7 mm inner diameter, 0.38 ml of transport medium is required in order to have a medium fill height of 10 mm and 0.96 ml of transport medium is required to provide a medium fill height of 25 mm.

It is notable that when the swab is inserted into the transport medium, it displaces a certain volume of transport medium. For example, in an experiment, a nasopharyngeal swab with a swab tip length of 23 mm and diameter of 3 mm was inserted into a container having an inner diameter of 6 mm and a length of 100 mm containing 0.4 ml of water, and was found to displace 0.06 ml of water. It is contemplated that a swab tip bearing a biological sample having a higher viscosity than water may have an even higher displacement volume. The displacement volume of transport medium in specimen transport medium tubes of other dimensions can be readily determined experimentally by the person of skill in the art without undue effort or the need of inventive skill. Therefore, taking this displaced volume into account, an even lower volume of transport medium than is listed above in Table 4 can be used to achieve the targeted medium fill height.

Thus, in at least one embodiment, the transport medium compartment has an inner diameter of less than or equal to 10 mm. In at least one embodiment, the transport medium compartment has an inner diameter of less than or equal to 8 mm. In at least one embodiment, the transport medium compartment has an inner diameter of less than or equal to 6 mm. In at least one embodiment, the transport medium compartment has an inner diameter of less than or equal to 5 mm. In at least one embodiment, the transport medium compartment has an inner diameter of less than or equal to 4 mm. Additionally, in at least one embodiment, the transport medium compartment has a length sufficient to accommodate the entire length of a swab tip. Thus, in at least one embodiment, the transport medium compartment has a length from about 10 mm to about 40 mm. In at one embodiment, the transport medium compartment has a length of about 30 mm. In at one embodiment, the transport medium compartment has a length of about 35 mm. In at least one embodiment, the transport medium compartment has a volume sufficient to accommodate a volume of transport medium less than or equal to 1 ml. In at least one embodiment, when a swab tip is fully inserted in the transport medium compartment filled with transport medium, at least 66% of the length of the swab tip is immersed in the transport medium. In at least one embodiment, when a swab tip is fully inserted in the transport medium compartment filled with transport medium, at least 90% of the length of the swab tip is immersed in the transport medium.

In at least one embodiment, the transport medium compartment contains a volume of transport medium which is less than the available volume inside the compartment, such that the compartment also contains a volume of air. In such embodiments, when a swab is inserted into the transport medium compartment, the volume of transport medium displaced by the swab can displace at least a portion of the air and be accommodated within the transport medium compartment. In at least one embodiment, the volume of air is from about 0.1 ml to about 0.2 ml. In at least one alternative embodiment, the transport medium compartment contains a volume of transport medium which is equal to or more than the available volume inside the compartment, such that the transport medium also occupies a portion of the tapered portion or the mouth portion of the specimen transport medium tube. In such embodiments, when a swab is inserted into the transport medium compartment, the volume of transport medium displaced by the swab will further occupy a portion of the tapered portion or the mouth portion of the specimen transport medium tube and the transport medium compartment will contain no air.

In at least one embodiment, the specimen transport medium tube can be manufactured from plastic, paper, glass or metal materials, as known in the art, and may be generally cylindrical or prismatic in shape with a round or polygonal cross-section. In at least one embodiment, the material from which the specimen transport medium tube is manufactured can be biodegradable. In at least one embodiment, the specimen transport medium tube can include markings to indicate the estimated volume of liquid contents of the tube. For example, markings can be etched or painted on the tube to indicate volumes including but not limited to 0.05 ml, 0.1 ml, 0.2 ml, 0.3 ml, 0.4 ml, 0.5 ml, 0.6 ml, 0.7 ml, 0.8 ml and 0.9 ml.

In at least one embodiment, the specimen transport medium tube is intended to be disposable so as to be discarded after a single use. In at least one embodiment, the specimen transport medium tube may be reusable. In such embodiments, a used specimen transport medium tube may be cleaned and sterilized using procedures well known in the art, including but not limited to autoclaving, irradiation with gamma radiation and treatment with ethylene oxide, and refilled with transport medium by the user of the specimen transport medium tube. In at least one embodiment, a clean or unused specimen transport medium tube may be filled with transport medium by a user and the filled tube may subsequently be sterilized so as to simultaneously sterilize the transport medium.

Thus, it is contemplated that the present specimen transport medium tube may be provided for use in a wide variety of embodiments. In at least one embodiment, the transport medium compartment of the specimen transport medium tube may be empty, so as to be fillable to a desired level with a desired transport medium by a user. In at least one embodiment, the transport medium compartment of the specimen transport medium tube can be pre-filled with transport medium by the manufacturer. In at least one embodiment, the specimen transport medium tube, including any transport medium contained within the tube, can be pre-sterilized by the manufacturer. In at least one embodiment, the specimen transport medium tube, whether or not pre-filled or pre-sterilized, can be pre-packaged by the manufacturer with a swab for collecting a sample. In at least one embodiment, the swab may be pre-sterilized. In at least one embodiment, the swab may be pre-attached to the cap and cap insert of the specimen transport medium tube as described herein. In at least one embodiment, the specimen transport medium tube, transport medium and swab may be provided together by the manufacturer as a kit, which may or may not be pre-sterilized. In at least one embodiment of the kit, the swab may be packaged separately from the specimen transport medium tube or may be pre-attached to the tube, which may be pre-filled with the transport medium or may be fillable with transport medium provided in a separate container within the kit. The kit may further contain instructions on the use of the components of the kit. Other configurations in which the present specimen transport medium tube may be provided for use will be evident to the user of skill in the art.

An additional aspect of the present invention provides a method for collecting swab samples or specimens for laboratory testing, including but not limited to microbiological, molecular diagnostic and other examinations. In at least one embodiment, the method includes using a swab comprising a swab handle and a swab tip as described herein to collect a sample on the swab tip. The swab tip is then inserted into an opening of a mouth portion of a specimen transport medium tube as described herein such that the swab tip enters an opening of a transport medium compartment and is immersed in transport medium contained in the transport medium compartment. The swab handle may optionally be broken at a breakpoint or cut at any desired point to form a shortened swab comprising the swab tip, so that the shortened swab can be accommodated inside the specimen transport medium tube.

A cap including a cap insert, which includes a cap insert head, is then secured to the opening of the mouth portion, such that the cap insert head acts to seal the transport medium within the transport medium compartment, as described herein. The handle of the shortened swab may be completely sealed inside the transport medium compartment. Alternatively, the handle of the swab or shortened swab may extend beside or through the cap insert head into the mouth portion of the specimen transport medium tube and may optionally be further secured against or within the cap insert, as described herein. In at least one embodiment, the swab may be attached to the cap or cap insert prior to collecting the sample.

The specimen transport medium tube may then be transported to a facility for testing or analysis of the collected sample. In at least one embodiment, to retrieve the sample, the cap and cap insert can be removed from the tube, and the swab or shortened swab can be removed and discarded. In embodiments where the swab is attached to the cap or cap insert, removing the cap and cap insert can act to also remove the swab. In at least one embodiment, the specimen transport medium tube may be agitated prior to removal of the swab or shortened swab, to facilitate transfer of the collected sample from the swab tip to the transport medium within the tube. Such agitation may be carried out by shaking, tapping or vibrating the tube containing the swab tip, or by stirring the swab tip within the transport medium. In at least one embodiment, the specimen transport medium tube may be agitated with a vortex mixer, as known in the art. Prior to being discarded, the swab tip may be pressed against the sides of the specimen transport medium tube to express transport medium remaining trapped in the fibres or pores of the swab tip.

The transport medium containing the collected sample can then be retrieved from the tube for testing. In at least one embodiment, the transport medium containing the collected sample can be retrieved from the transport medium compartment by suctioning or aspirating the transport medium containing the collected sample from the tube. In at least one embodiment, a pipette tip attached to a squeeze bulb or to a commercially available mechanical or electronic pipettor can be inserted through the opening in the mouth portion of the specimen transport medium tube and the transport medium containing the collected sample can be suctioned into the pipette tip. In at least one embodiment, a slender pipette tip having an external diameter in the range of less than 1 mm to about 5 mm is conveniently used for this purpose, as such pipette tips can be inserted into the complete length of the transport medium compartment. Suitable pipette tips include but are not limited to Corning® gel-loading pipet tips having a volume of 1-200 μL and a diameter 0.5 mm, Fisherbrand™ Gel-Loading Tips, having a volume of 1-200 μL and an external diameter of 0.6 mm, and other known or commercially available pipette tips having a volume of 200 μl or less. It is advantageous that the larger diameter of the mouth portion of the specimen transport medium tube compared to the diameter of the transport medium compartment allows a user to more easily insert a pipette tip into the mouth portion of the tube.

In at least one embodiment where the specimen transport medium tube includes an opening at the distal end capped with a distal end cap, the transport medium containing the collected sample can be retrieved from the transport medium compartment by removing the distal end cap and allowing the transport medium containing the collected sample to drain into a collection vessel, which can be, for example, a microcentrifuge tube having a volume of 1.5 ml or 2 ml or a 15 ml or 50 ml conical tube, as is known in the art. Drainage of the transport medium containing the collected sample into the collection vessel can be facilitated by, for example, positioning a pipette tip mounted on a mechanical or electronic pipettor within the mouth portion of the tube and depressing the plunger of the pipettor, thus pushing air into the transport medium compartment through the pipette tip and forcing the transport medium through the opening at the distal end of the transport medium compartment into the collection vessel. A standard pipette tip having a volume of 1 ml or 200 μl is conveniently used for this purpose. The swab or shortened swab may be pushed out of the transport medium compartment along with the transport medium, or may be removed from the transport medium compartment before drainage of the transport medium. Prior to discarding the removed swab tip, the swab tip may be further stirred or agitated within the retrieved transport medium, to facilitate further transfer of the sample from the swab tip to the transport medium, and the swab tip can be pressed against the sides of the container holding the transport medium to express transport medium remaining trapped in the fibres or pores of the swab tip.

Advantageously, in at least one embodiment, a swab tip bearing a collected sample may remain immersed in a lower volume of transport medium during transport in the present specimen transport medium tube than in a conventional transport medium tube. In this way, the sample on the swab tip may be diluted to a lesser extent than in a conventional transport medium tube and may be protected from degradation and dehydration during transport. Thus, use of the present specimen transport medium tube may result in improved detection of low levels of an analyte in a sample, by quantitative polymerase chain reaction (qPCR) or quantitative reverse transcriptase PCR (qRT PCR) techniques, for example, without requiring a change in assay protocols or reagent concentrations. For example, the use of samples collected in the present specimen transport medium tube may enable pooling of samples. Alternatively, the use of samples collected in the present specimen transport medium tube may enable analysis while avoiding the need for RNA extraction. Thus, test cost and turnaround time may be reduced and throughput increased. In addition, use of the present specimen transport medium tube may find wide application in many areas, such as in environmental and industrial settings, where swab samples containing low levels of a variety of analytes might be collected.

### DESCRIPTION OF SPECIFIC EMBODIMENTS

Other features of the present invention will become apparent from the following non-limiting embodiments which illustrate, by way of example and with reference to the attached drawings, the principles of the invention.

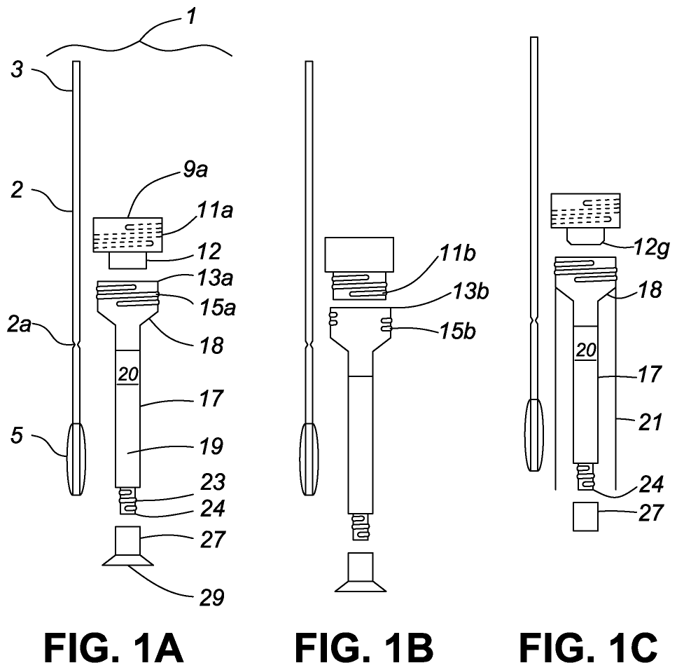

An embodiment of a sample collection kit according to the present application is shown in FIG. 1A. The kit 1 comprises a swab 3 having a swab handle 2 with breakpoint 2a and a swab tip 5. The kit 1 also comprises a specimen transport medium tube 17 having a mouth portion 13a and a transport medium compartment 19 having a reduced diameter compared to the diameter of mouth portion 13a, and containing transport medium 20. A tapered portion 18 with a gradually decreasing diameter connects mouth portion 13a to transport medium compartment 19. Cap 9a includes on its interior surface a helical screw thread 11 a which mates with complementary screw thread 15a on the exterior surface of mouth portion 13a. Cap 9a further includes cap insert 12, which snugly seats against the inner side walls of tapered portion 18 when cap 9a is secured to mouth portion 13a. Distal end cap 27, bearing support structure 29, also includes a helical screw thread which mates with complementary screw thread 23 on the exterior of the distal end 24 of specimen transport medium tube 17.

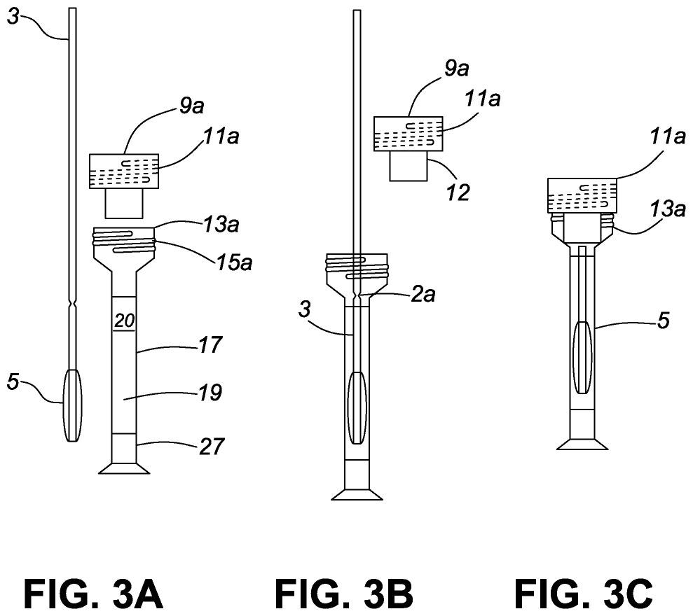

In use of the embodiment of FIG. 1A, as illustrated in FIGS. 3A-3C, swab 3 is used to collect a sample, such as an oropharyngeal sample, on swab tip 5. The swab 3 is then inserted in specimen transport medium tube 17 so that the swab tip 5 is immersed in transport medium 20 contained in transport medium compartment 19, as seen in FIG. 3B. The swab handle is then broken at breakpoint 2a to provide a shortened swab 3 which can be accommodated within specimen transport medium tube 17, and cap 9a with cap insert 12 is attached to mouth portion 13a and secured by engaging complementary helical screw threads 11a and 15a, so that cap insert 12 seals the opening of transport medium compartment 19 and secures shortened swab 3 within transport medium compartment 19, as seen in FIG. 3C.

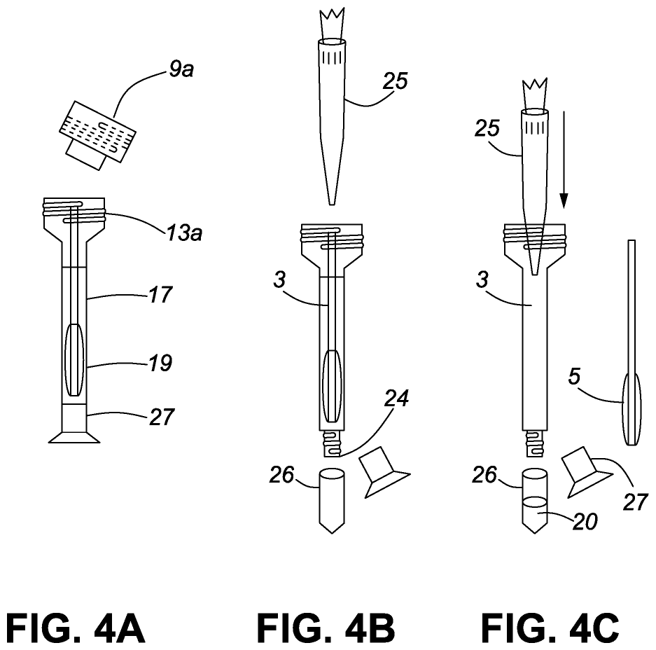

Once specimen transport medium tube 17 has been received in a testing facility, for example, the sample may be retrieved for testing as illustrated in FIGS. 4A-4C. Cap 9a is removed from mouth portion 13a, as seen in FIG. 4A and distal end cap 27 is removed from distal end 24 of specimen transport medium tube 17, as seen in FIG. 4B. Pipette tip 25 is inserted into mouth portion 13a and a pipettor attached to pipette tip 25 is used to push air into transport medium compartment 19, so as to push transport medium 20 out of transport medium compartment 19 into collection vessel 26, as seen in FIG. 4C. Swab 3 can be pushed out of transport medium compartment 19 into collection vessel 26 along with transport medium 20, or alternatively, swab 3 can be removed from transport medium compartment 19 with tweezers, for example, before the removal of transport medium 20 from transport medium compartment 19.

An alternative embodiment of the present sample collection kit is shown in FIG. 1B, in which cap insert 12 bears an external helical screw thread 11b which mates with complementary screw thread 15b situated on the interior of mouth portion 13b. Another alternative embodiment, shown in FIG. 1C, includes a cap insert 12 having a contoured cap insert head 12g which complements the internal contour of tapered portion 18 so as to form a snug fit therewith. The embodiment shown in FIG. 1C also includes external side wall 21 which extends to the distal end 24 of the specimen transport medium tube 17, providing a support structure for the tube. External side wall 21 is open at distal end 24, allowing ready removal and replacement of distal end cap 27, so that transport medium compartment 19 can be accessed from distal end 24.

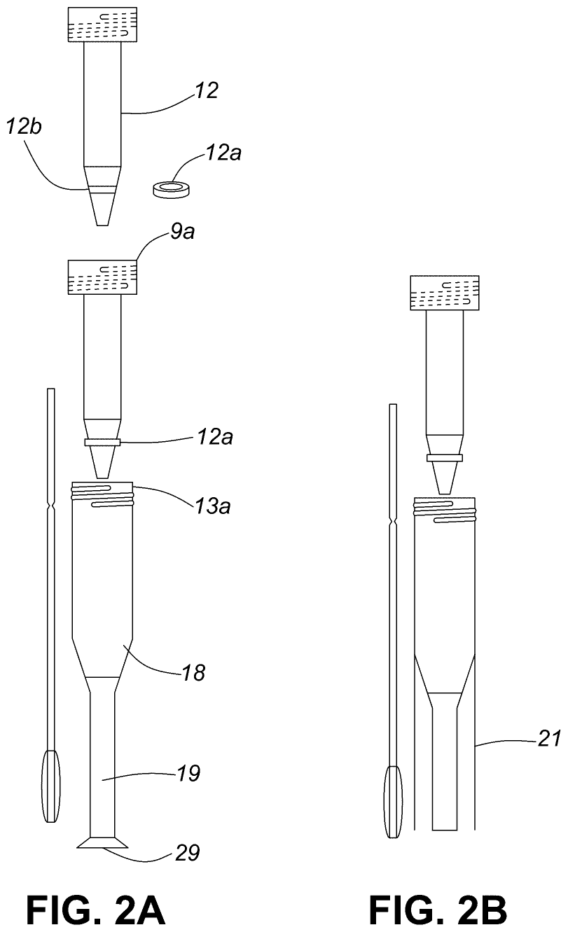

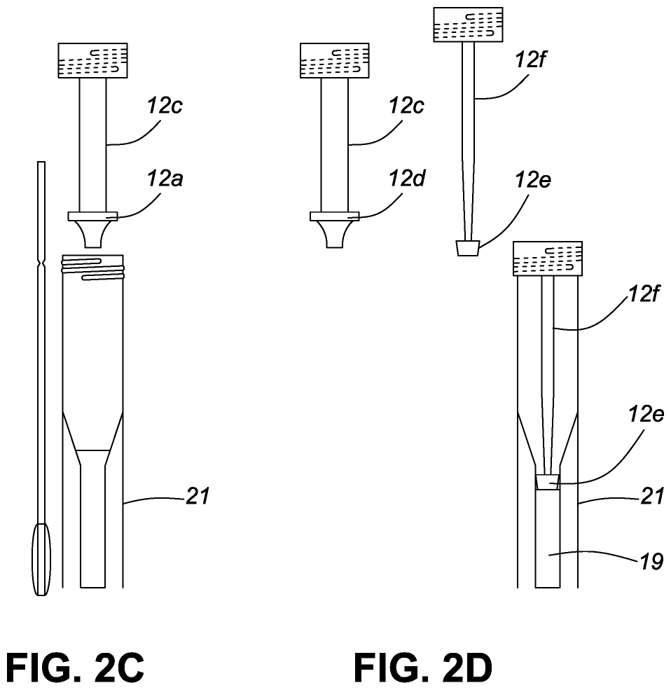

FIG. 2A shows an embodiment of the present sample collection kit similar to that of FIG. 1A, but in which the specimen transport medium tube 17 has a longer mouth portion 13a configured so that specimen transport medium tube 17 may accommodate a swab or a shortened swab with a longer net length after breakage at the breakpoint, while keeping the volume of transport medium compartment 19 advantageously low, as discussed above. In this embodiment, the cap insert 12 is conically shaped at the end distal from the cap, so as to complement the internal contour of tapered portion 18. Cap insert 12 also bears a cap insert head in the form of O-ring 12a fitted into groove 12b, and has a length such that O-ring 12a can seat snugly against the inner side wall of tapered portion 18 when the cap 9a is secured to mouth portion 13a of tube 17. FIG. 2B shows an embodiment similar to that of FIG. 2A but containing external side wall 21, similar to the embodiment of FIG. 1C.

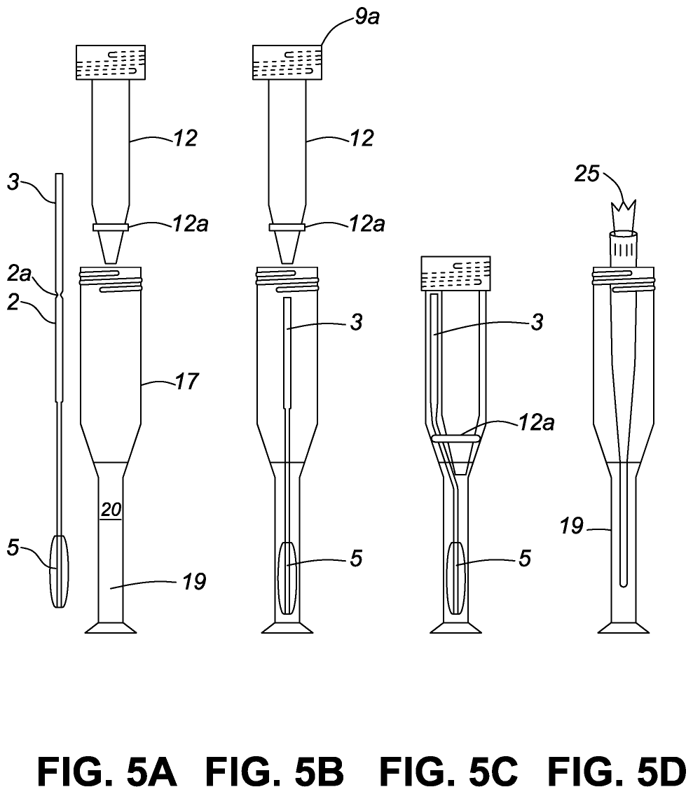

In use of the embodiment of FIG. 2A, as illustrated in FIGS. 5A to 5D, swab 3, having a flexible handle 2, is used to collect a sample, such as a nasopharyngeal sample, on swab tip 5. The swab 3 is then inserted in specimen transport medium tube 17 so that the swab tip 5 is immersed in transport medium 20 contained in transport medium compartment 19, as seen in FIG. 5B. The swab handle is then broken at breakpoint 2a to provide a shortened swab 3 which can be accommodated within specimen transport medium tube 17. The flexibility of swab handle 2 allows the insertion of cap 9a and cap insert 12 into specimen transport medium tube 17 to displace the handle 2 towards the side wall of tube 17. When cap 9a is secured to the mouth portion of specimen transport medium tube 17, O-ring 12a on cap insert 12 snugly seats against the sloped side wall of tube 17, deforming to accommodate the width of handle 2 and sealing the opening of transport medium compartment 19, as seen in FIG. 5C. When the specimen transport medium tube 17 is received in a testing facility, the cap 9a and the shortened swab 3 can be removed from the tube and transport medium 20 containing the sample can be suctioned out for analysis using pipette tip 25.

FIG. 2C shows an alternative embodiment in which O-ring 12a is attached to a cap insert 12c which has a smaller diameter than cap insert 12 of the embodiment of FIG. 2A, thereby reducing the cost of the material needed to manufacture the cap insert while preserving the ability of the O-ring 12a to seat snugly against the inner side wall of tapered portion 18. As seen in FIG. 2D, the cap insert head 12d can have a generally conical shape contoured to seat snugly against the side wall of tapered portion 18. In addition, alternative cap insert head 12e can form a seal within the opening of transport medium compartment 19, allowing cap insert 12f to have an even smaller diameter than cap insert 12c.

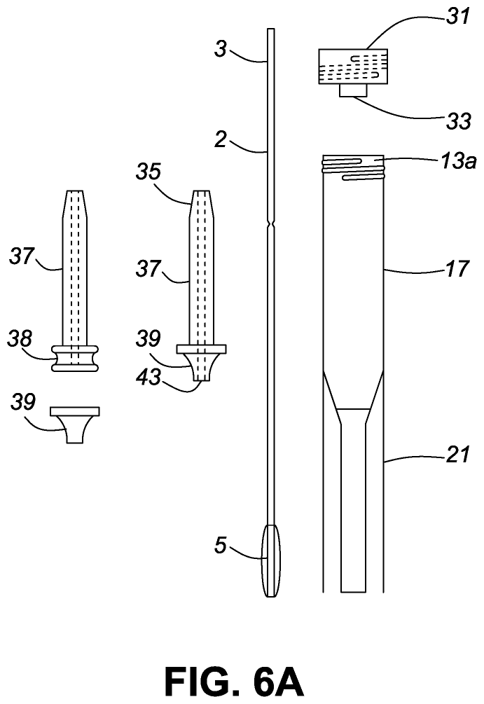

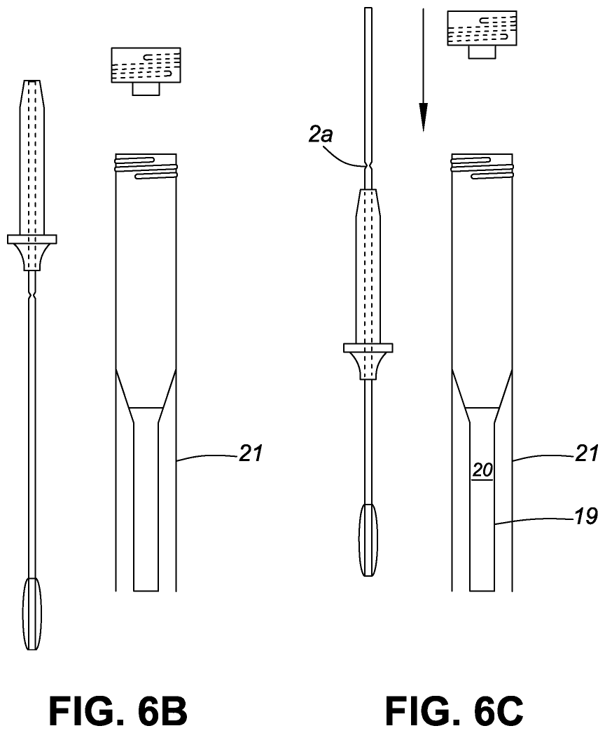

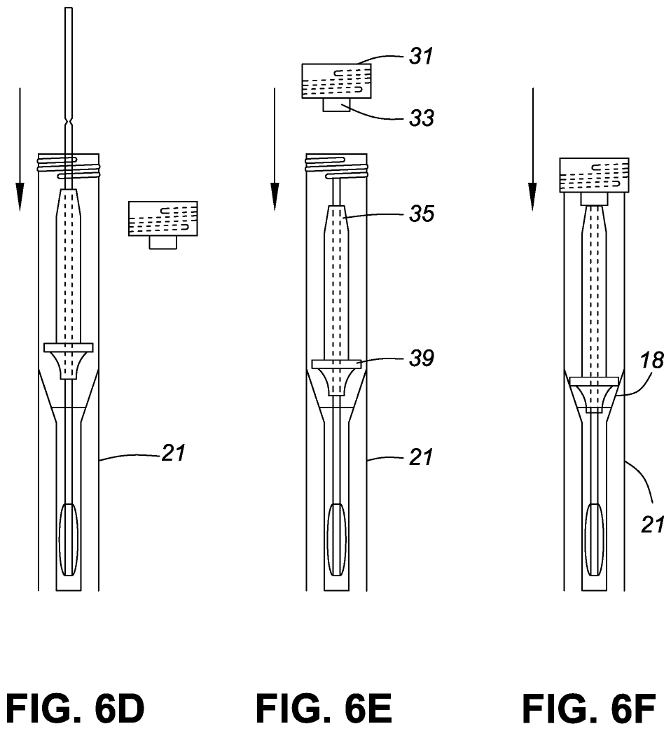

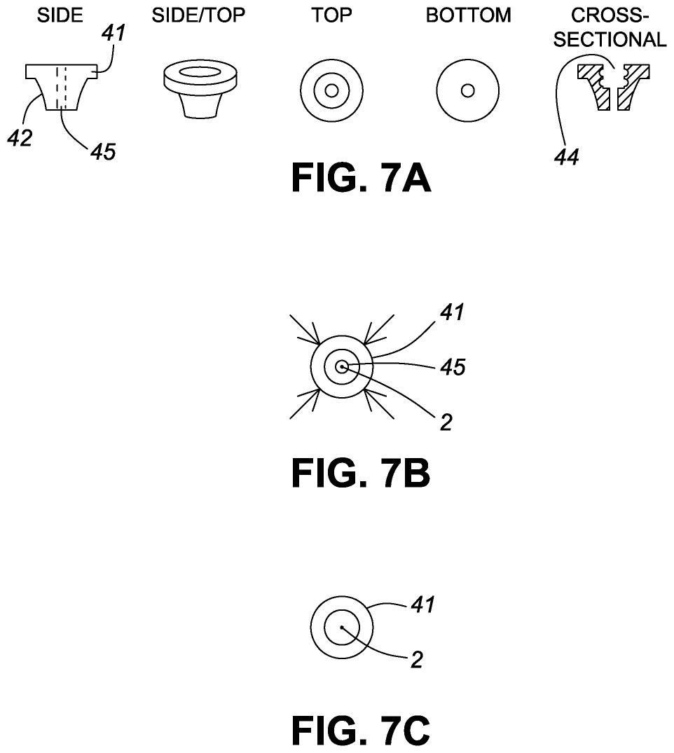

An alternative embodiment of the present sample collection kit is described with reference to FIGS. 6A to 6F and 7A to 7C, including specimen transport medium tube 17 with external side wall 21 as previously described, cap 31 and cap insert 37. Cap 31 contains a helical screw thread to engage a complementary helical screw thread at the mouth portion 13a of specimen transport medium tube 17, as previously described. Cap insert 37 has a first end 35, which is configured to connect to connector 33 on cap 31. Cap insert 37 also has a second end 38 bearing ridges configured to engage recess 44 in cap insert head 39, as seen in FIG. 7A, by interlocking or with a snug friction fit. In at least one embodiment, the ridges at second end are manufactured from a plastic material, including but not limited to polypropylene or polystyrene. Cap insert 37 further contains an internal longitudinal channel 43 configured to snugly accept swab handle 2 of swab 3. In at least one embodiment, cap insert 37 is perforated or includes openings perpendicular to internal longitudinal channel 43, through which swab handle 2 may be visible when inserted into internal longitudinal channel 43. As seen in FIG. 7A, cap insert head 39 includes sealing portion 41, conical portion 42 and interior channel 45, which, like internal longitudinal channel 43 of cap insert 37, is configured to snugly accept swab handle 2 of swab 3. In at least one embodiment, the diameter of sealing portion 41 is larger than the largest internal diameter of tapered portion 18 of specimen transport medium tube 17. In at least one embodiment, the diameter of conical portion 42 is larger than the internal diameter of a corresponding point on tapered portion 18.

In use, as seen in FIGS. 6A, 6B and 6C, the swab 3 is used to collect a sample on swab tip 5, and swab handle 2 is inserted into interior channel 45 of cap insert head 39 (seen in FIG. 7A) and internal longitudinal channel 43 of cap insert 37 until breakpoint 2a on swab handle 2 passes through the length of internal longitudinal channel 43 and emerges at first end 35 of cap insert 37. The swab 3 and cap insert 37 are then inserted into specimen transport medium tube 17 so that swab tip 5 is immersed in transport medium 20 located in transport medium compartment 19, as seen in FIG. 6D. Swab handle 2 is then broken at breakpoint 2a so that the length of shortened swab 3 is accommodated within specimen transport medium tube 17, as seen in FIG. 6E. In an alternative embodiment, swab 3 may be pre-inserted in cap insert 37, either by a user of the specimen collection kit or by a manufacturer of the kit, before the swab is used to collect a sample.

As cap 31 engages the mouth portion of tube 17, connector 33 engages first end 35 of cap insert 37 with a snap fit connection as known in the art (Christopher M. Schlick (3 Oct. 2009). Industrial Engineering and Ergonomics: Visions, Concepts, Methods and Tools Festschrift in Honor of Professor Holger Luczak. Springer Science & Business Media. pp. 597-. ISBN 978-3-642-01293-8, and Henry W. Stoll (1 Jun. 1999). Product Design Methods and Practices. CRC Press. pp. 172—. ISBN 978-0-8247-7565-0.) to connect cap 31 to cap insert 37 and shortened swab 3, as seen in FIG. 6F. Once connector 33 has engaged first end 35 of cap insert 37 to connect cap 31 to cap insert 37, removal of cap 31 from specimen transport medium tube 17 will also act to remove cap insert 37 and shortened swab 3 from specimen transport medium tube 17, facilitating removal of the transport medium containing the sample from specimen transport medium tube 17.

In at least one embodiment, the diameter of sealing portion 41 is larger, for example by about 2% to about 15%, than the largest internal diameter of tapered portion 18 of specimen transport medium tube 17. In at least one embodiment, the diameter of conical portion 42 at any given point is larger, for example by about 2% to about 15%, than the internal diameter of a corresponding point on the internal side wall of tapered portion 18. Thus, as cap 31 is tightened onto the mouth portion of specimen transport medium tube 17, cap insert 37 is inserted further into the specimen transport medium tube 17, resiliently compressing and snugly seating sealing portion 41 of cap insert head 39 against the inner side wall of tapered portion 18 of specimen transport medium tube 17, thereby sealing transport medium 20 inside transport medium compartment 19. Furthermore, as illustrated in FIGS. 7B and 7C, the compression of cap insert head 39 also compresses interior channel 45 around the swab handle 2, such that the material of cap insert head 39 snugly seals to swab handle 2, preventing any loss of transport medium 20 through interior channel 45.

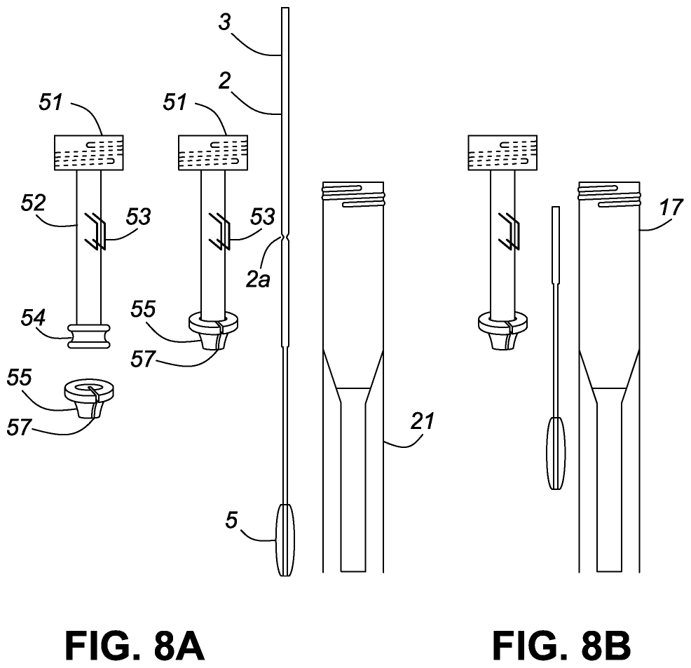

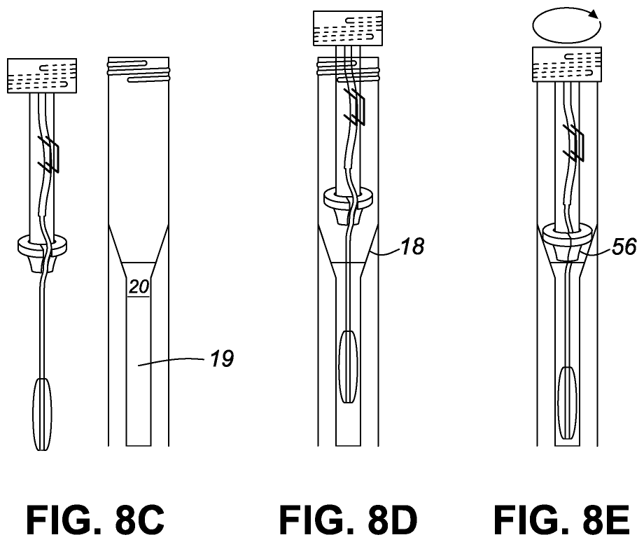

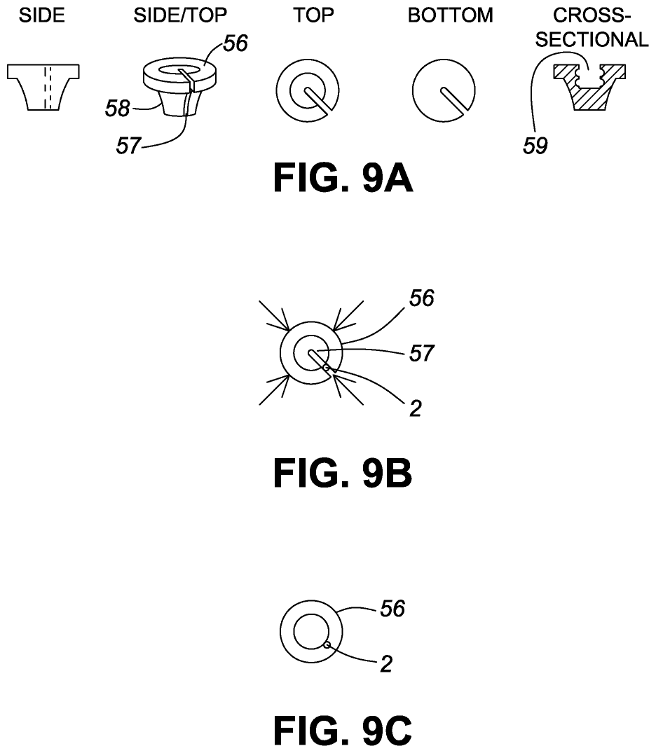

A further alternative embodiment of the present sample collection kit is described with reference to FIGS. 8A to 8E and 9A to 9C, including specimen transport medium tube 17 with external side wall 21 as previously described, cap 51 and cap insert 52. Cap 51 and cap insert 52 may be unitary in construction, or may be two separate pieces assembled and fixed together, for example, during manufacturing of the sample collection kit. Cap insert 52 includes bracket 53 which is configured to receive and retain flexible swab handle 2. Cap insert 52 also includes cap insert head 55, as further seen in FIG. 9A, which includes sealing portion 56 and conical portion 58, and is similar to cap insert head 39 of FIGS. 6A to 6F. Cap insert head 55 can be attached to cap insert 52 by inserting ridges 54 on cap insert 52 into recess 59 of cap insert head 55, seen in FIG. 9A. However, cap insert head 55 includes a longitudinal slit 57 in place of an interior channel. In at least one embodiment, cap insert head 55 can include more than one longitudinal slit. In at least one embodiment, the longitudinal slit or slits extend radially from the edge of sealing portion 56 towards the interior of cap insert head 55 a distance of no more than half of the radius of cap insert head 55. In at least one embodiment, the longitudinal slit or slits extend radially from the edge of sealing portion 56 towards the interior of cap insert head 55 a distance of no more than one-third of the radius of cap insert head 55. In this way, ridges 54 on cap insert 52 may be securely retained in recess 59 of cap insert head 55.

In use, as seen in FIG. 8B, the swab 3 is used to collect a sample on swab tip 5, and swab handle 2 is broken at breakpoint 2a so that the length of shortened swab 3 can be accommodated within specimen transport medium tube 17, as seen in FIG. 8B. The flexible swab handle 2 is then inserted into longitudinal slit 57 and into bracket 53, as seen in FIG. 8C. The insertion of swab handle 2 into longitudinal slit 57 is facilitated when cap insert head includes more than one longitudinal slit, as the flexibility of sealing portion 56 is increased. Because swab handle 2 is attached to cap 51 and cap insert 52 by its insertion into longitudinal slit 57 and bracket 53, removal of cap 51 from specimen transport medium tube 17 will also act to remove cap insert 52 and shortened swab 3 from specimen transport medium tube 17, facilitating removal of the transport medium containing the sample from specimen transport medium tube 17.

The shortened swab 3 and cap insert 52 are then inserted into specimen transport medium tube 17 so that swab tip 5 is immersed in transport medium 20 located in transport medium compartment 19, as seen in FIG. 8D. Securing cap 51 onto the mouth portion of specimen transport medium tube 17, acts to insert cap insert 52 further into the specimen transport medium tube 17, resiliently compressing and snugly seating sealing portion 56 of cap insert head 55 against the inner side wall of tapered portion 18 of specimen transport medium tube 17, thereby sealing transport medium 20 inside transport medium compartment 19, as seen in FIG. 8E. Furthermore, as illustrated in FIGS. 9B and 9C, the compression of cap insert head 55 also compresses longitudinal slit 57 around the swab handle 2, such that the material of cap insert head 55 snugly seals to swab handle 2, preventing any loss of transport medium 20 through longitudinal slit 57. Thus, in at least one embodiment, the diameter of sealing portion 56 is larger, for example by about 2% to about 15%, than the largest internal diameter of tapered portion 18 of specimen transport medium tube 17. In at least one embodiment, the diameter of conical portion 58 at any given point is larger, for example by about 2% to about 15%, than the internal diameter of a corresponding point on the internal side wall of tapered portion 18.

### EXAMPLES

Other features of the present invention will become apparent from the following non-limiting examples which illustrate, by way of example, the principles of the invention.

### Example 1

### Comparative Test for Leakage and Swab Immersion in Transport Medium

One unit of a specimen transport medium tube as described herein was opened by screwing open the cap. A nasopharyngeal swab was retrieved by opening the packing pouch of the swab. The swab stick was shortened by breaking the stick at the breakpoint. The swab with the shortened swab stick (net swab stick) was put in the specimen transport medium tube, and the transport medium compartment was filled with 0.3 ml of a blue food dye solution. The cap which was pre-assembled with the cap insert was replaced onto the tube and the cap was screwed onto the tube.

The cap insert head was observed to surround not only the swab stick immediately next to the swab, but also to be in contact to the tapered portion of the tube, such that the seal of the liquid inside the transport medium compartment was complete. It was further observed that when inverted, the swab in the specimen transport medium tube was fully immersed in the transport media. By contrast, the swab when placed in a conventional transport medium tube (Disposable Sampler, 10 mL vial commercially available from NEST Biotechnology Co., Ltd, catalogue no. 202092) filled with 0.3 ml of dye solution was not fully immersed in the transport media before the tube was inverted. When the tube was inverted, the swab in the control tube was not immersed. The present specimen transport medium tube ensured the immersion of the swabs at all times in transport medium and no leakage of the liquid was observed from the transport medium compartment of the tube to the main compartment of the tube. This sealing and lack of leakage was observed for 5 days, even with the tube inverted upside down.

### Example 2

### Comparative Test for Analyte Concentration in Transport Medium

Eighteen specimen transport medium tubes as described herein along with 18 conventional tubes were each filled with transport medium (Hank's based balance salt based, non-inactivating medium, 0.3 ml for the present specimen transport medium tubes and 3 ml for the conventional tubes). Each of 36 swabs was dipped, one by one, into a spiked mock sample containing a SARS-CoV-2 RNA (COVID-19 viral RNA). Then each of 18 dipped swabs was placed into a separate present specimen transport medium tube; each of the other 18 swabs was placed into a separate conventional tube. Aliquots of 100 μl of the transport medium from each of the 36 tubes were removed, heated and then tested in a duplex SARS-CoV-2 quantitative reverse transcription PCR. The duplex SARS-CoV-2 quantitative reverse transcription PCR tested 2 regions (N1 and N2 regions) of the viral SARS-CoV-2 RNA to further validate the assay results. The Cq values from the quantitative reverse transcription PCR are shown in Table 5 below.

TABLE 5
Cq results for N1 and N2 genes from samples collected in conventional
tubes and present specimen transport medium tubes

| | N1 Gene | | N2 Gene | |
|---|---|---|---|---|
| Replicate # | Conventional | Present tube | Conventional | Present tube |
| 1 | 32.9 | 30.1 | 36.5 | 30.7 |
| 2 | 34.0 | 29.2 | 37.7* | 30.2 |
| 3 | 33.1 | 29.5 | 35.5 | 36.4** |
| 4 | 32.7 | 29.6 | 36.0 | 30.7 |
| 5 | 32.0 | 29.3 | 37.8* | 30.4 |
| 6 | 32.4 | 29.6 | 42.7* | 30.3 |
| 7 | 33.2 | 29.4 | NaN⁺ | 31.0 |
| 8 | 32.5 | 29.4 | NaN⁺ | 29.8 |
| 9 | 32.0 | 29.5 | NaN⁺ | 30.6 |
| 10 | 31.8 | 29.7 | 38.3* | 30.3 |
| 11 | 32.7 | 29.8 | 35.9 | 31.3 |
| 12 | 33.0 | 29.8 | 35.3 | 31.4 |
| 13 | 34.1 | 29.3 | NaN⁺ | 30.0 |
| 14 | 32.4 | 29.4 | NaN⁺ | 30.2 |
| 15 | 31.3 | 29.4 | 34.3 | 30.1 |
| 16 | 32.3 | 29.9 | 34.6 | 30.7 |
| 17 | 32.9 | 29.6 | 36.1 | 30.4 |
| 18 | 33.1 | 30.0 | 35.4 | 30.8 |
| Average Cq | 32.7 | 29.6 | 35.5* | 30.9 |
| Coefficient of | 2.2% | 0.8% | 2.0% | 1.5%** |
| variation (CV) | | | | |
| % | | | | |

Notes:
*Cq values >37 were excluded from the calculation of average Cq and CV.
⁺NaN—Not detected.
**CV calculation excluded the outlier value of 36.4 in this group.

As seen from the results shown in Table 5, the average Cq value for the N1 gene quantitative (real-time) PCR for the present specimen transport medium tube was 29.6, which was 3.1 less than that for the conventional tube (average Cq 32.7). Since Cq is a direct reflection of the analyte concentration, this difference corresponds to 8.5-fold more viral RNA analyte concentration in the present specimen transport medium tube than in the conventional tube. The difference of average Cq value for N2 gene between the two groups of tubes was 4.7 (average Cq 35.5 for the conventional tube and 30.9 for the present specimen transport medium tube), corresponding to a 25-fold viral RNA analyte concentration in the present specimen transport medium tube compared to that in the conventional tube. The results from both N1 and N2 regions suggest that a higher viral RNA analyte concentration is found in the transport media contained in the present specimen transport medium tubes, reflecting the reduced volume of the transport medium in the present specimen transport medium tube compared to that in the conventional tube.

Since the volumes of transport media were 0.3 ml for the present specimen transport medium tube and 3 ml for the conventional tube respectively, the concentration of the analytes eluted from the swab into the collected sample in transport medium in the present specimen transport medium tube was expected to be 10-fold higher compared to that in the conventional tube. The observed increase in viral RNA analyte concentration of greater than 10-fold for the N2 test may be attributed to the inherently greater errors expected when Cq values approach 37.

In addition, it was observed that the presence of viral RNA was not detectable in 5 of 18 samples in the conventional tube group, whereas the presence of viral RNA was detected in all 18 samples from the present specimen transport medium tubes. Therefore, it can be concluded from the results of this study that the detection of low concentrations of analyte in samples is significantly improved by using the present specimen transport medium tubes.

### Example 3

### Comparative Test of Nasopharyngeal Swab Samples

Twenty previously tested and anonymized nasopharyngeal swab samples obtained during COVID-19 testing which had been stored in universal transport medium (UTM; Hanks Balanced Salt, non-inactivating type) in a freezer at -80° C. were used in this study. A local research ethics board review and approval were obtained prior to the study. Based on initial SARS-COV-2 N-gene testing results, 7 samples had Cq values of <25, 6 samples had Cq values of 25-30, 6 samples had Cq values of 30-35, 1 sample had Cq values of 35-40.

0.1 ml aliquots of these 20 samples, which had previously been found positive for containing the SARS-COV-2 gene, were spiked to each of a present specimen transport medium tube containing 0.3 ml of UTM and a paired control viral transport media (VTM) tube containing 3 mL of UTM, to provide two sets of 20 tubes each. The UTMs containing the samples in each tube were then retrieved and subjected to viral RNA extraction by KingFisher™ Nucleic Acid Isolation system (ThermoFisher) and quantitative reverse transcription PCR test, using a kit supplied by ThermoFisher. Because of the dilutions and the loss of viral titers due to the freeze thaw process, some of the positive samples were expected to become undetectable in this study. The results are shown in Tables 6 to 9 below.

TABLE 6
Detecting patient samples in the present specimen
transport medium tubes and the control tubes
using SARS-COV-2 N and ORF1ab genes

| | Cq value | | | | | |
|---|---|---|---|---|---|---|
| | N-gene | | | ORF1ab | | |
| Patient | Present | Control | Delta | Present | Control | Delta |
| Sample ID | Tubes | VTM | Cq | Tubes | VTM | Cq |
| 1 | 18.7 | 21.1 | 2.4 | 18.4 | 20.7 | 2.3 |
| 2 | 21.5 | 24.4 | 2.9 | 21.2 | 24.2 | 3 |
| 3 | 25.1 | 27.4 | 2.3 | 24.7 | 27.4 | 2.7 |
| 4 | 29.7 | 30.9 | 1.2 | 29.6 | 30.6 | 1 |
| 5 | 33.9 | 34.8 | 0.9 | 33.0 | 33.6 | 0.6 |
| 6 | ND | ND | N.A. | 39.2 | 38.7 | −0.5 |
| 7 | 25.0 | 27.4 | 2.4 | 24.7 | 27.4 | 2.7 |
| 8 | 28.5 | 30.7 | 2.2 | 28.4 | 30.5 | 2.1 |
| 9 | 31.1 | 34.5 | 3.4 | 31.0 | 33.5 | 2.5 |
| 10 | ND | ND | N.A. | 34.9 | 38.9 | 4 |
| 11 | 28.4 | 37.3 | 8.9 | 29.9 | 35.9 | 6 |
| 12 | ND | ND | N.A. | 38.3 | ND | N.A. |
| 13 | 32.7 | 39.4 | 6.7 | 31.7 | 34.2 | 2.5 |
| 14 | 29.5 | 32.5 | 3 | 29.4 | 32.6 | 3.2 |
| 15 | ND | ND | N.A. | 37.1 | ND | N.A. |
| 16 | ND | ND | N.A. | 38.2 | ND | N.A. |
| 17 | 27.6 | 30.8 | 3.2 | 27.8 | 31.4 | 3.6 |
| 18 | 24.8 | 27.5 | 2.7 | 24.7 | 27.6 | 2.9 |
| 19 | ND | ND | N.A. | ND | ND | N.A. |
| 20 | ND | ND | N.A. | ND | ND | N.A. |

Note:
ND—not detected.

TABLE 7
Number of positives and sensitivity of detection for the present specimen transport
medium tube and control tube groups using SARS-COV-2 N and ORF1ab Genes

| | Number of Positives (sensitivity) | | | | | |
|---|---|---|---|---|---|---|
| | N-gene | | | ORF1ab | | |
| Cq | Present | Control | Gain in | Present | Control | Gain in |
| Cut-off | Tubes | VTM | sensitivity | Tubes | VTM | sensitivity |
| Cq <40 | 13(65%) | 13(65%) | 0 | 18(90%) | 15(75%) | 15% |
| Cq <38 | 13(65%) | 12(60%) | 5% | 15(75%) | 13(65%) | 10% |
| Cq <36 | 13(65%) | 11(55%) | 10% | 14(70%) | 13(65%) | 5% |

TABLE 8
Detecting patient samples in the present specimen transport medium tube
and the control tubes using E-gene, RDRP and Human RNaseP Tests

| | Cq Value | | | | | | | | |
|---|---|---|---|---|---|---|---|---|---|
| Patient | E-gene | | | RDRP | | | RNaseP | | |
| Sample | Present | Control | Delta | Present | Control | Delta | Present | Control | Delta |
| ID | Tubes | VTM | Cq | Tubes | VTM | Cq | Tubes | VTM | Cq |
| 1 | 19.3 | 33.2 | 13.9 | 18.7 | 33.2 | 14.5 | 31.7 | 27.8 | 3.9 |
| 2 | 22.0 | 25.6 | 3.6 | 22.7 | 25.3 | 2.6 | 33.5 | 32.0 | 1.5 |
| 3 | 26.2 | 28.4 | 2.2 | 26.0 | 28.8 | 2.8 | 33.5 | 33.4 | 0.1 |
| 4 | 30.3 | 31.7 | 1.4 | 30.9 | 31.7 | 0.8 | 31.7 | 27.8 | 3.9 |
| 5 | 33.2 | 35.2 | 2.0 | 33.2 | 34.4 | 1.2 | 31.7 | 28.0 | 3.7 |
| 6 | 28.2 | 38.6 | 10.4 | 37.3 | 37.5 | 0.2 | 36.3 | 31.7 | 4.6 |
| 7 | 26.0 | ND | | 25.5 | 28.0 | 2.5 | 33.2 | 29.3 | 3.9 |
| 8 | 29.6 | 32.6 | 3.0 | 29.0 | 32.3 | 3.3 | 33.2 | 33.2 | 0.0 |
| 9 | 32.2 | 33.2 | 1.0 | 33.3 | 33.2 | −0.1 | 31.2 | 28.0 | 3.2 |
| 10 | 36.4 | 38.0 | 1.6 | 37.5 | ND | | 33.5 | 27.0 | 6.5 |
| 11 | 30.9 | 36.7 | 5.7 | 31.8 | 38.1 | 6.3 | 29.6 | 27.5 | 2.1 |
| 12 | ND | ND | | ND | ND | | 33.1 | 30.3 | 2.8 |
| 13 | 33.0 | 36.9 | 4.0 | 33.0 | 36.8 | 3.8 | 35.9 | 32.8 | 3.1 |
| 14 | 30.1 | 33.8 | 3.7 | 30.3 | 33.6 | 3.3 | 32.6 | 28.4 | 4.2 |
| 15 | ND | ND | | 38.4 | ND | | 34.9 | 30.2 | 4.7 |
| 16 | ND | ND | | ND | ND | | 35.3 | 30.7 | 4.6 |
| 17 | 29.0 | 32.3 | 3.3 | 29.1 | 32.2 | 3.2 | 34.8 | 30.2 | 4.6 |
| 18 | 25.8 | 28.6 | 2.8 | 25.9 | 28.5 | 2.6 | 37.6 | 34.4 | 3.2 |
| 19 | 39.5 | ND | | ND | ND | | 32.6 | 29.0 | 3.6 |
| 20 | ND | ND | | ND | ND | | 32.7 | 28.0 | 4.7 |

Note:
ND—not detected.

TABLE 9
Number of positives and sensitivity of detection for the present specimen transport
medium tube and control groups using SARS-COV-2 E-gene, RDRP and human RNaseP tests

| | Number of Positives (sensitivity) | | | | | | | | |
|---|---|---|---|---|---|---|---|---|---|
| | E-gene | | | RDRP | | | RNaseP | | |
| | | | Gain in | | | Gain in | | | Gain in |
| Cq | Present | Control | sensi- | Present | Control | sensi- | Present | Control | sensi- |
| Cut-off | Tubes | VTM | tivity | Tubes | VTM | tivity | Tubes | VTM | tivity |
| Cq <40 | 16 | 14 | 10% | 16 | 14 | 10% | 20 | 20 | 0% |
| | (80%) | (70%) | | (80%) | (70%) | | (100%) | (100%) | |
| Cq <38 | 15 | 13 | 10% | 15 | 13 | 10% | 20 | 20 | 0% |
| | (75%) | (65%) | | (75%) | (65%) | | (100%) | (100%) | |
| Cq <36 | 14 | 10 | 15% | 14 | 11 | 15% | 20 | 18 | 10% |
| | (70%) | (50%) | | (70%) | (55%) | | (100%) | (90%) | |

The results (Tables 6 and 8) showed that the Cq values for the five gene markers tested for the present specimen transport medium tube were lower than those for the control tubes, as expected. It is notable that the expected difference in dilution factors (0.1 ml into 0.4 ml or 0.1 ml into 3.1 mL) was 7.75-fold, which corresponds to a Cq difference of 2.95. Thus, the observed differences in Cq between the two types of tubes were consistent with this expected difference.

Tables 7 and 9 show the sensitivity values of detection for all five genes. Sensitivity clearly showed better values with the present specimen transport medium tube than with the control tube, with a gain of sensitivity around 10%.

In summary, in this example, the present specimen transport medium tube group showed consistently better (lower) Cq values than the control group. The lower Cq values were associated with better sensitivity of detection among samples prepared in the present specimen transport medium tubes compared to samples prepared in the control tubes.

As used herein, the terms “about” or “approximately” as applied to a numerical value or range of values are intended to mean that the recited values can vary within an acceptable degree of error for the quantity measured given the nature or precision of the measurements, such that the variation is considered in the art as equivalent to the recited values and provides the same function or result. For example, the degree of error can be indicated by the number of significant figures provided for the measurement, as is understood in the art, and includes but is not limited to a variation of ±1 in the most precise significant figure reported for the measurement. Typical exemplary degrees of error are within 20 percent (%), preferably within 10%, and more preferably within 5% of a given value or range of values. Alternatively, and particularly in biological systems, the terms “about” and “approximately” can mean values that are within an order of magnitude, preferably within 5-fold and more preferably within 2-fold of a given value. Numerical quantities given herein are approximate unless stated otherwise, meaning that the term “about” or “approximately” can be inferred when not expressly stated.

As used herein, the term “substantially” refers to the complete or nearly complete extent or degree of an action, characteristic, property, state, structure, item, or result. For example, a quantity that has a value “substantially” equal to the value of another quantity has so nearly the same value, within an acceptable degree of error, that the quantities provide the same function or result. The exact allowable degree of deviation from absolute completeness may in some cases depend on the specific context. However, generally speaking the nearness of completion will be so as to have the same overall result as if absolute and total completion were obtained.

The use of “substantially” is equally applicable when used in a negative connotation to refer to the complete or near complete lack of an action, characteristic, property, state, structure, item, or result. For example, a composition that is “substantially free of” an ingredient or element would either completely lack that ingredient or element, or so nearly completely lack that ingredient or element that the effect would be the same as if it completely lacked that ingredient or element. In other words, a composition that is “substantially free of” an ingredient or element may still actually contain such item as long as there is no measurable or significant effect thereof.

As used herein, terms indicating relative direction or orientation, including but not limited to “upper”, “lower”, “top”, “bottom”, “vertical”, “horizontal”, “outer”, “inner”, “front”, “back”, and the like, are intended to facilitate description of the present invention by indicating relative orientation or direction in usual use, and are not intended to limit the scope of the present invention in any way to such orientations or directions.

The embodiments described herein are intended to be illustrative of the present compositions and methods and are not intended to limit the scope of the present invention. Various modifications and changes consistent with the description as a whole and which are readily apparent to the person of skill in the art are intended to be included. The appended claims should not be limited by the specific embodiments set forth in the examples, but should be given the broadest interpretation consistent with the description as a whole.

1. A specimen transport medium tube for transporting a sample disposed on a swab tip of a swab, the specimen transport medium tube comprising:
a mouth end comprising an opening configured to receive the swab tip;
a cap configured to engage the mouth end so as to seal the opening, the cap comprising a cap insert;
a distal end opposite the mouth end and defining a longitudinal axis therewith; and
at least one side wall extending longitudinally between the mouth end and the distal end to define and enclose an interior space inside the specimen transport medium tube;
wherein the interior space is in fluid communication with the opening of the mouth end and wherein the interior space comprises:
a mouth portion at the mouth end having an inner diameter thereof;
a transport medium compartment longitudinally aligned with the longitudinal axis and configured to contain a transport medium and the swab tip, the transport medium compartment having an inner diameter thereof and an inner volume thereof, wherein the inner diameter of the transport medium compartment is smaller than the inner diameter of the mouth portion and is less than or equal to 10 mm, and the inner volume of the transport medium compartment is less than 1 ml; and
a tapered portion disposed between the mouth portion and the transport medium compartment, wherein the tapered portion is in fluid communication with the mouth portion and with the transport medium compartment, and is configured to permit unhindered passage of the swab tip from the opening at the mouth end to the transport medium compartment,
wherein an inner diameter of the tapered portion decreases from the inner diameter of the mouth portion at a junction between the tapered portion and the mouth portion to the inner diameter of the transport medium compartment at a junction between the tapered portion and the transport medium compartment;
wherein the cap insert is configured to prevent fluid communication between the mouth portion and the transport medium compartment when the cap engages the mouth end.

2. The specimen transport medium tube according to claim 1, wherein the distal end comprises a distal opening in fluid communication with the transport medium compartment and wherein the specimen transport medium tube further comprises a distal cap configured to engage the distal end so as to seal the distal opening.

3. The specimen transport medium tube according to claim 1, wherein the distal end comprises a distal end wall aligned perpendicular to the one or more side walls and unitary therewith.

4. The specimen transport medium tube according to claim 1 further containing a support structure configured to stably support the specimen transport medium tube at rest on the distal end thereof.

5. The specimen transport medium tube according to claim 1 further comprising one or more external side walls longitudinally aligned with the longitudinal axis and external of at least the one or more side walls at the tapered portion and at the transport medium compartment.

6. The specimen transport medium tube according to claim 5 wherein the specimen transport medium tube is configured for use in an automated liquid handling system.

7. The specimen transport medium tube according to claim 1 wherein the cap insert is configured to engage a handle of the swab.

8. The specimen transport medium tube according to claim 1 further including the transport medium within the transport medium compartment.

9. The specimen transport medium tube according to claim 1 wherein the inner diameter of the transport medium compartment is less than or equal to 8 mm.

10. The specimen transport medium tube according to claim 1 wherein the inner diameter of the transport medium compartment is less than or equal to 5 mm.

11. The specimen transport medium tube according to claim 1 wherein the inner volume of the transport medium compartment is less than or equal to 0.8 ml.

12. The specimen transport medium tube according to claim 1 wherein the inner volume of the transport medium compartment is less than or equal to 0.5 ml.

13. The specimen transport medium tube according to claim 1 wherein the cap insert forms a seal against the one or more side walls of the tapered portion when the cap engages the mouth end.

14. A specimen collection kit comprising a swab; a transport medium; and a specimen transport medium tube as defined in claim 1.

15. The specimen collection kit according to claim 14 further containing instructions for use of the kit.

16. The specimen collection kit according to claim 14 wherein the transport medium is contained in the transport medium compartment of the specimen transport medium tube.

17. The specimen collection kit according to claim 14 wherein the swab is affixed to the cap of the specimen transport medium tube.

18. The specimen collection kit according to claim 14 wherein the swab is a nasopharyngeal swab.

19. The specimen collection kit according to claim 14 wherein the swab is an oropharyngeal swab.
